(19) United States
(12) Patent Application Publication
(10) Pub. No.: US 2024/0225448 A1
(43) Pub. Date: Jul. 11, 2024

(54) CHARACTERISING LESIONS IN THE LIVER USING DYNAMIC CONTRAST-ENHANCED MAGNETIC RESONANCE TOMOGRAPHY

(71) Applicant: Bayer Aktiengesellschaft, Leverkusen (DE)

(72) Inventors: Gunnar SCHUETZ, Berlin (DE); Gesine KNOBLOCH, Berlin (DE)

(73) Assignee: Bayer Aktiengesellschaft, Leverkusen (DE)

(21) Appl. No.: 18/559,323

(22) PCT Filed: Apr. 28, 2022
(86) PCT No.: PCT/EP2022/061289

§ 371 Date: Nov. 6, 2023

(30) Foreign Application Priority Data
May 7, 2021 (EP) 21172677.3

(51) Int. Cl. A61B 5/00; A61B 5/055; A61K 49/10; G01R 33/56

(52) U.S. Cl. CPC A61B 5/004; A61B 5/055; A61B 5/4244; A61K 49/103; G01R 33/5601; G01R 33/5608

(57) ABSTRACT

The invention relates to the technical field of characterising lesions in the liver using dynamic contrast-enhanced magnetic resonance tomography.

### CROSS-REFERENCE TO RELATED APPLICATIONS

This application is a national stage application under 35 U.S.C. § 371 of International Application No. PCT/EP2022/061289, filed internationally on Apr. 28, 2022, which claims the benefit of priority to European Application No. 21172677.3, filed on May 7, 2021.

### FIELD

The present invention deals with the technical field of characterization of lesions in the liver by means of dynamic contrast-enhanced magnetic resonance imaging.

### BACKGROUND

The liver can be affected by a plurality of benign tumours, which can appear as cystic or solid focal lesions in the liver parenchyma. However, the liver is also vulnerable to malignant tumours such as metastases of extrahepatic types of cancer or of primary types of cancer that have their origin in the liver itself. Globally, the two most common types of malignant liver tumours are metastases—especially metastases of bowel cancer—and hepatocellular carcinomas (HCC). Almost 20% of bowel cancer patients have liver metastases at the time of diagnosis, and more than 50% of bowel cancer patients develop liver metastases in the course of their disease. Hepatocellular carcinoma (HCC) is the most common primary liver cancer. It is also the sixth most common cancer worldwide and the fourth most common cause of cancer-related deaths.

The accurate and reliable detection and characterization of focal liver lesions in early disease stages is of great clinical relevance, especially in the case of patients at risk of liver metastases or primary liver cancer, since they are of fundamental importance for appropriate planning of treatment and determine the suitability of potentially curative treatment options.

Magnetic resonance imaging (MRI) is of particular importance for the radiological examination of liver lesions. It is distinguished by outstanding contrast of soft tissues and high spatial resolution without exposing the patient to ionizing radiation or iodinated contrast agents.

The contrast agents most commonly used in MRI are paramagnetic contrast agents based on gadolinium. These agents are administered via an intravenous (i.v.) bolus injection. Their contrast-enhancing effect is mediated by the central gadolinium ion (Gd-III) in the chelate complex. If T1-weighted (w) scanning sequences are used in MRI, the gadolinium ion-induced shortening of the spin-lattice relaxation time (T1) of excited atomic nuclei leads to an increase in the signal intensity and hence to an increase in the image contrast of the tissue examined.

From their pattern of spreading in tissue, gadolinium-based contrast agents can be roughly divided into extracellular and intracellular contrast agents.

Extracellular contrast agents refer to low-molecular-weight, water-soluble compounds which, after intravenous administration, spread in the blood vessels and in the interstitial space. After a certain, comparatively short period of circulation in the blood circulation system, they are excreted via the kidneys. The extracellular MRI contrast agents include, for example, the gadolinium chelates gadobutrol (Gadovist®), gadoteridol (Prohance®), gadoteric acid (Dotarem®), gadopentetic acid (Magnevist®) and gadodiamide (Omnican®).

Intracellular contrast agents are taken up into the cells of tissues to a certain extent and subsequently excreted. Intracellular MRI contrast agents based on gadoxetic acid are, for example, distinguished by the fact that they are proportionately specifically taken up by liver cells, the hepatocytes, accumulate in the functional tissue (parenchyma) and enhance the contrasts in healthy liver tissue before they are subsequently excreted via the gallbladder into the faeces. Examples of such contrast agents based on gadoxetic acid are described in U.S. Pat. No. 6,039,931A; they are commercially available for example under the trade names Primovist® and Eovist®. A further MRI contrast agent having a lower uptake into the hepatocytes is gadobenate dimeglumine (Multihance®).

Gadoxetate disodium (GD, Primovist®) belongs to the group of intracellular contrast agents. It is authorized for use in MRI of the liver for detecting and characterizing lesions in patients with known or suspected focal liver disease. With its lipophilic ethoxybenzyl unit, GD exhibits two-phase spreading: spreading at first in the intravascular and interstitial space after bolus injection, followed by selective uptake by hepatocytes. GD is excreted from the body unaltered via the kidneys and the hepatobiliary route (50:50 dual mechanism of excretion) in about the same amounts. Because of its selective accumulation in healthy liver tissue, GD is also referred to as a hepatobiliary contrast agent.

GD is authorized in a dose of 0.1 ml/kg of body weight (BW) (0.025 mmol/kg BW Gd). The recommended administration of GD comprises an undiluted intravenous bolus injection at a flow rate of about 2 ml/second, followed by flushing of the i.v. cannula with a physiological saline solution. A standard protocol for liver imaging using GD consists of multiple planning and pre-contrast sequences. After i.v. bolus injection of the contrast agent, dynamic images are usually acquired during the arterial phase (about 30 seconds after the injection, p.i.), portal venous phase (about 60 seconds p.i.) and transitional phase (about 2-5 minutes p.i.). Typically, the transitional phase already shows a certain rise in the liver signal intensity owing to the incipient uptake of the agent into hepatocytes. Additional T2-weighted and diffusion-weighted (DWI) images can be generated after the dynamic phase and before the late hepatobiliary phase.

The contrast-enhanced dynamic images from the arterial phase, portal venous phase and transitional phase provide crucial information about the patterns of lesion enhancement (vascularization) that vary over time and that contribute to characterization of the specific liver lesion. Hepatocellular carcinoma, with its typical arterial phase hyperenhancement (APHE) and the washout of the contrast in the venous phase, can be diagnosed solely on the basis of its unique vascularization pattern, which is observed during dynamic phase imaging, and patients are protected from an invasive and possibly risky liver biopsy as a result.

Other lesions can also be characterized using dynamic contrast-enhanced MRI.

In the diagnosis of liver lesions, the advantage of a hepatobiliary contrast agent over an extracellular contrast agent is that it has a higher sensitivity and hence allows better detection of relatively small carcinomas in particular (see, for example: R. F. Hanna et al.: Comparative 13-year meta-analysis of the sensitivity and positive predictive value of ultrasound, CT, and MRI for detecting hepatocellular carcinoma, Abdom Radiol 2016, 41, 71-90; Y. J. Lee et al.: Hepatocellular carcinoma: diagnostic performance of multidetector CT and MR imaging—a systematic review and meta-analysis, Radiology 2015, 275, 97-109; D. K Owens et al.: High-value, cost-conscious health care: concepts for clinicians to evaluate the benefits, harms, and costs of medical interventions, Ann Intern Med 2011, 154, 174-180).

The problem is, then, that the dynamic increase and/or decrease in contrast in liver lesions is commonly assessed by visual inspection by radiologists (subjective assessment of the relative change in contrast between liver tissue and liver lesion); misinterpretations may occur as a result.

In the case of an assessment by visual inspection, whether an extracellular or an intracellular contrast agent is used does make a difference. For example, when using a hepatobiliary contrast agent, an increase in signal in healthy liver tissue may be misinterpreted as a washout of contrast agent from neighbouring liver lesions (relative increase in contrast between liver tissue and liver lesion).

Therefore, the European Association for the Study of Liver has, in its guidelines (EASL Clinical Practice Guidelines: Management of hepatocellular carcinoma), specified different features for the identification of a hepatocellular carcinoma depending on the contrast agent used: (see Journal of Hepatology, 2018, Vol. 69, pages 182-236):
when using an extracellular contrast agent: combination of hypervascularity in the late arterial phase and washout in the portal venous phase and/or the delayed phase,
when using a hepatobiliary contrast agent: combination of hypervascularity in the late arterial phase and washout in the portal venous phase.

Thus, whereas the use of an extracellular contrast agent for detection of hepatocellular carcinomas involves using both the portal venous phase and the subsequent delayed phase, use of a hepatobiliary contrast agent involves using only the portal venous phase for detection of a washout and not using a phase subsequent to the portal venous phase.

As already explained, the reason therefor is that a hepatobiliary contrast agent (in contrast to an extracellular contrast agent) is taken up by the liver cells, where it accumulates before it is excreted via the gallbladder into the faeces. Thus, when using a hepatobiliary contrast agent, an initial comparatively rapid vascular contrast enhancement in the arterial phase is followed by a contrast enhancement in healthy liver tissue that further increases slowly and continuously. If a radiologist visually compares the contrast enhancement in a lesion with the contrast enhancement in healthy liver tissue, the continuously increasing contrast enhancement in healthy liver tissue may be misinterpreted as a washout of contrast agent from lesions.

Thus, it is explicitly recommended by the European Association for the Study of Liver that, when using a hepatobiliary contrast agent, the analysis of MRI images for identification of a hepatocellular carcinoma be restricted to the arterial and the portal venous (up to 60 seconds after the intravenous administration of the contrast agent).

However, the problem is that some lesions can be unambiguously characterized only by the dynamic behaviour in a later phase.

In the case of some hepatocellular carcinomas, washout becomes apparent, for example, only after the portal venous phase (see, for example, C. J. Zech et al.: Consensus report from the 8th International Forum for Liver Magnetic Resonance Imaging, European Radiology 2020, 30, 370-382).

If the time span after the portal venous phase is, as recommended by the European Association for the Study of Liver, not taken into account for the detection of a washout, then, for example, those hepatocellular carcinomas for which the washout becomes detectable only after the portal venous phase remain undetected. The consequence may be that more biopsies must be carried out in order to check whether a lesion is a benign lesion or a malignant tumour (see especially FIG. 2 in Journal of Hepatology, 2018, Vol. 69, page 194). A biopsy is not just additional effort for medical personnel; it also means a risk to the patient.

It would be desirable to be able to reduce the number of such biopsies. It would be desirable to be able to identify and characterize liver lesions reliably without the risk, when using a hepatobiliary contrast agent, of a rise in signal of healthy liver tissue being misinterpreted as a washout of contrast agent from liver lesions.

This is achieved by the present invention.

### SUMMARY

Provided is a computer-implemented method for identifying a washout of contrast agent from a region of a liver of a patient during a dynamic contrast-enhanced magnetic resonance imaging examination, comprising the steps of:
receiving a plurality of representations,
   wherein the plurality of representations represents
      the liver of the patient or part of the liver of the patient, and
      reference tissue of the patient,
   wherein at least one representation of the plurality of representations represents
      the liver or the part of the liver, and
      the reference tissue
   during a portal venous phase of the dynamic contrast-enhanced magnetic resonance imaging examination, and
   wherein at least one representation of the plurality of representations represents
      the liver or the part of the liver, and
      the reference tissue
   during a transitional phase of the dynamic contrast-enhanced magnetic resonance imaging examination,
identifying one or more regions in the liver
   in which contrast agent leads in the portal venous phase and/or the transitional phase to a lower contrast enhancement than in the reference tissue, and/or
   in which the contrast enhancement in the portal venous phase and/or the transitional phase drops more rapidly than in the reference tissue, wherein the reference tissue does not comprise hepatocytes, and/or
   in which the absolute value of the gradient of the decreasing contrast enhancement in the portal venous phase and/or the transitional phase is greater than the absolute value of the gradient of the increasing contrast enhancement in healthy liver tissue,
outputting a representation of the liver or the part of the liver, wherein in the representation the identified region is highlighted or the identified regions are highlighted.

Additionally, described is a computer system comprising:
a receiving unit,
a control and calculation unit, and
an output unit,
   wherein the control and calculation unit is configured to prompt the receiving unit to receive a plurality of representations,
      wherein the plurality of representations represents
         a liver of a patient or part of the liver of the patient, and
         reference tissue of the patient,
      wherein at least one representation of the plurality of representations represents
         the liver or the part of the liver, and
         the reference tissue
      during a portal venous phase of a dynamic contrast-enhanced magnetic resonance imaging examination, and
      wherein at least one representation of the plurality of representations represents
         the liver or the part of the liver, and
         the reference tissue
      during a transitional phase of a dynamic contrast-enhanced magnetic resonance imaging examination,
wherein the control and calculation unit is configured to identify one or more regions in the liver
   in which contrast agent leads in the portal venous phase and/or the transitional phase to a lower contrast enhancement than in the reference tissue, and/or
   in which the contrast enhancement in the portal venous phase and/or the transitional phase drops more rapidly than in the reference tissue, wherein the reference tissue does not comprise hepatocytes, and/or
   in which the absolute value of the gradient of the decreasing contrast enhancement in the portal venous phase and/or the transitional phase is greater than the absolute value of the gradient of the increasing contrast enhancement in healthy liver tissue,
wherein the control and calculation unit is configured to prompt the output unit to output a representation of the liver or the part of the liver, wherein in the representation the identified region is highlighted or the identified regions are highlighted.

A computer program product is also provided. The computer program product may comprise a computer program which can be loaded into a memory of a computer, where it prompts the computer to execute the following steps:
receiving a plurality of representations,
   wherein the plurality of representations represents
      a liver of a patient or part of the liver of the patient, and
      reference tissue of the patient,
   wherein at least one representation of the plurality of representations represents
      the liver or the part of the liver, and
      the reference tissue
   during a portal venous phase of a dynamic contrast-enhanced magnetic resonance imaging examination, and
   wherein at least one representation of the plurality of representations represents
      the liver or the part of the liver, and
      the reference tissue
   during a transitional phase of a dynamic contrast-enhanced magnetic resonance imaging examination,
identifying one or more regions in the liver
   in which contrast agent leads in the portal venous phase and/or the transitional phase to a lower contrast enhancement than in the reference tissue, and/or
   in which the contrast enhancement in the portal venous phase and/or the transitional phase drops more rapidly than in the reference tissue, wherein the reference tissue does not comprise hepatocytes, and/or
   in which the absolute value of the gradient of the decreasing contrast enhancement in the portal venous phase and/or the transitional phase is greater than the absolute value of the gradient of the increasing contrast enhancement in healthy liver tissue,
outputting a representation of the liver or the part of the liver, wherein in the representation the identified region is highlighted or the identified regions are highlighted.

The present invention further provides for the use of a contrast agent in a dynamic magnetic resonance imaging examination method, wherein the examination method comprises the following steps:
administering the contrast agent,
generating a plurality of representations,
   wherein the plurality of representations represents
      a liver of a patient or part of the liver of the patient, and
      reference tissue of the patient,
   wherein at least one representation of the plurality of representations represents
      the liver or the part of the liver, and
      the reference tissue
   during a portal venous phase of the examination method, and
   wherein at least one representation of the plurality of representations represents
      the liver or the part of the liver, and
      the reference tissue
   during a transitional phase of the examination method,
identifying one or more regions in the liver
   in which contrast agent leads in the portal venous phase and/or the transitional phase to a lower contrast enhancement than in the reference tissue, and/or
   in which the contrast enhancement in the portal venous phase and/or the transitional phase drops more rapidly than in the reference tissue, wherein the reference tissue does not comprise hepatocytes, and/or
   in which the absolute value of the gradient of the decreasing contrast enhancement in the portal venous phase and/or the transitional phase is greater than the absolute value of the gradient of the increasing contrast enhancement in healthy liver tissue,
outputting a representation of the liver or the part of the liver, wherein in the representation the identified region is highlighted or the identified regions are highlighted.

The present invention further provides a contrast agent for use in a dynamic contrast-enhanced magnetic resonance imaging examination method, wherein the examination method comprises the following steps:
administering the contrast agent,
generating a plurality of representations,
   wherein the plurality of representations represents
      a liver of a patient or part of the liver of the patient, and
      reference tissue of the patient,
   wherein at least one representation of the plurality of representations represents
      the liver or the part of the liver, and
      the reference tissue
   during a portal venous phase of the examination method, and
   wherein at least one representation of the plurality of representations represents
      the liver or the part of the liver, and
      the reference tissue
   during a transitional phase of the examination method,
identifying one or more regions in the liver
   in which contrast agent leads in the portal venous phase and/or the transitional phase to a lower contrast enhancement than in the reference tissue, and/or
   in which the contrast enhancement in the portal venous phase and/or the transitional phase drops more rapidly than in the reference tissue, wherein the reference tissue does not comprise hepatocytes, and/or
   in which the absolute value of the gradient of the decreasing contrast enhancement in the portal venous phase and/or the transitional phase is greater than the absolute value of the gradient of the increasing contrast enhancement in healthy liver tissue,
outputting a representation of the liver or the part of the liver, wherein in the representation the identified region is highlighted or the identified regions are highlighted.

The present invention further provides a kit comprising a contrast agent and the computer program product according to the invention.

Further subjects of the invention and preferred embodiments of the invention are found in the dependent claims, in the present description and in the drawings.

### BRIEF DESCRIPTION OF THE FIGURES

The invention will be described, by way of example only, with reference to the following figures.

FIG. 1 shows the temporal profile (t=time) of the signal intensities I that are caused by a hepatobiliary contrast agent in liver arteries (A), liver veins (V) and healthy liver cells (L) in a dynamic contrast-enhanced MRI examination, according to some embodiments.

FIGS. 2(a)-2(b) show graphs of hyperenhancement and hypoenhancement, according to some embodiments.

FIG. 3 shows representations of the liver of a patient at various time points, according to some embodiments.

FIG. 4 shows the representations of the liver of a patient depicted in FIG. 3 at a smaller scale, according to some embodiments.

FIG. 5 shows a computer system, according to some embodiments.

### DETAILED DESCRIPTION

The invention will be more particularly elucidated below without distinguishing between the subjects of the invention (method, computer system, computer program product, use, contrast agent for use, kit). On the contrary, the following elucidations are intended to apply analogously to all the subjects of the invention, irrespective of in which context (method, computer system, computer program product, use, contrast agent for use, kit) they occur.

The present invention provides means for automatic identification of a washout of contrast agent from a region of a liver of a patient during a dynamic contrast-enhanced magnetic resonance imaging examination. In other words: the present invention makes it possible to automatically identify a region or multiple regions within the liver that are characterized by a washout of contrast agent.

Washout refers to the observation that the contrast enhancement in a region of a liver in the portal venous phase and/or the transitional phase of a dynamic contrast-enhanced magnetic resonance imaging examination drops more rapidly than in the surrounding (healthy) liver tissue.

Such a washout is often used as a characterizing feature for specification of liver lesions (see, for example: Y. I. Liu et al.: Quantitatively Defining Washout in Hepatocellular Carcinoma, American Journal of Roentgenology 2013 200:1, 84-89; Journal of Hepatology, 2018, Vol. 69, pages 182-236).

Magnetic resonance imaging, MRI for short, is an imaging method which is used especially in medical diagnostics for depicting structure and function of the tissues and organs in the human or animal body.

In MRI, the magnetic moments of protons in an examination object are aligned in a basic magnetic field, with the result that there is a macroscopic magnetization along a longitudinal direction. This is then deflected from the resting position by irradiation with high-frequency pulses (excitation). The return of the excited states to the resting position (relaxation), or magnetization dynamics, is then detected as relaxation signals by means of one or more high-frequency receiver coils.

For spatial encoding, rapidly switched magnetic gradient fields are superimposed on the basic magnetic field. The captured relaxation signals, or detected and spatially resolved MRI data, are initially present as raw data in a spatial frequency space, and can be transformed by subsequent Fourier transform into real space (image space).

In the case of native MRI, the tissue contrasts are created by the different relaxation times (T1 and T2) and the proton density.

T1 relaxation describes the transition of the longitudinal magnetization to its equilibrium state, T1 being the time taken to reach 63.21% of the equilibrium magnetization prior to the resonance excitation. It is also called longitudinal relaxation time or spin-lattice relaxation time.

T2 relaxation describes in an analogous manner the transition of transverse magnetization to its equilibrium state.

In a first step of a dynamic contrast-enhanced magnetic resonance imaging examination, an MRI contrast agent is administered to an examination object.

The “examination object” is usually a living being, preferably a mammal, very particularly preferably a human. The term “patient” is also used in this description.

The contrast agent can be an extracellular or an intracellular contrast agent. Preference is given to a hepatobiliary contrast agent.

A hepatobiliary contrast agent is understood to mean a contrast agent which is specifically taken up by healthy liver cells, the hepatocytes.

Examples of hepatobiliary contrast agents are contrast agents based on gadoxetic acid. They are, for example, described in U.S. Pat. No. 6,039,931A. They are commercially available under the trade names Primovist® or Eovist® for example.

The contrast-enhancing effect of Primovist®/Eovist® is mediated by the stable gadolinium complex Gd-EOB-DTPA (gadolinium ethoxybenzyl diethylenetriaminepentaacetic acid). DTPA forms with the paramagnetic gadolinium ion a complex that has extremely high thermodynamic stability. The ethoxybenzyl (EOB) radical is the mediator of the hepatobiliary uptake of the contrast agent.

In a particularly preferred embodiment, the contrast agent used is a substance or a substance mixture having gadoxetic acid or a salt of gadoxetic acid as contrast-enhancing active substance. Very particular preference is given to the disodium salt of gadoxetic acid (Gd-EOB-DTPA disodium).

After the intravenous administration of the hepatobiliary contrast agent in the form of a bolus into an arm vein, the contrast agent reaches the liver first via the arteries. These are depicted with contrast enhancement in the corresponding MRI images. The phase in which the liver arteries are depicted with contrast enhancement in MRI images is referred to as “arterial phase”.

Subsequently, the contrast agent reaches the liver via the liver veins. Whereas the contrast in the liver arteries is already decreasing, the contrast in the liver veins is reaching a maximum. The phase in which the liver veins are depicted with contrast enhancement in MRI images is referred to as “portal venous phase”. Said phase can already start during the arterial phase and overlap therewith.

The portal venous phase is followed by the “transitional phase”, in which the contrast in the liver arteries drops further and the contrast in the liver veins likewise drops. When using a hepatobiliary contrast agent, the contrast in the healthy liver cells gradually rises in the transitional phase.

The arterial phase, the portal venous phase and the transitional phase are also referred to collectively as “dynamic phase”.

10-20 minutes after its injection, a hepatobiliary contrast agent leads to a distinct signal enhancement in the healthy liver parenchyma. This phase is referred to as “hepatobiliary phase”. The contrast agent is eliminated only slowly from the liver cells; accordingly, the hepatobiliary phase can last for two hours and longer.

The stated phases are, for example, described in more detail in the following publications: J. Magn. Reson. Imaging, 2012, 35(3): 492-511, doi:10.1002/jmri.22833; Clujul Medical, 2015, Vol. 88 no. 4: 438-448, DOI: 10.15386/cjmed-414; Journal of Hepatology, 2019, Vol. 71: 534-542, http://dx.doi.org/10.1016/j.jhep.2019.05.005).

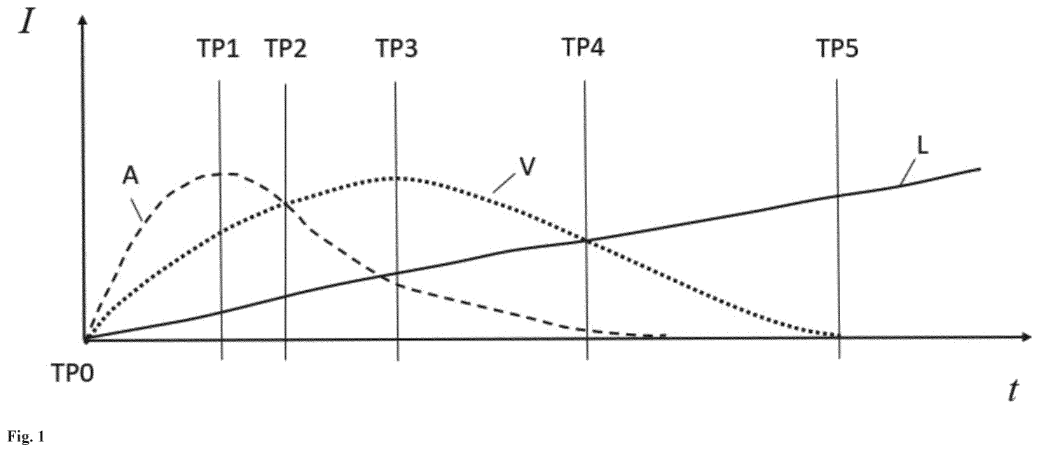

FIG. 1 shows schematically the temporal profile (t=time) of the signal intensities I that are caused by a hepatobiliary contrast agent in liver arteries (A), liver veins (V) and healthy liver cells (L) in a dynamic contrast-enhanced MRI examination. The signal intensity I has a positive correlation with the concentration of the contrast agent in the stated regions. Upon an intravenous bolus injection, the concentration of the contrast agent rises in the liver arteries (A) first of all (dashed curve). The concentration passes through a maximum and then drops. The concentration in the liver veins (V) rises more slowly than in the liver arteries and reaches its maximum later (dotted curve). The concentration of the contrast agent in the liver cells (L) rises slowly (solid curve) and reaches its maximum only at a very much later time point (not depicted in FIG. 1). A few characteristic time points can be defined: At time point TP0, contrast agent is administered intravenously as a bolus. Since the administration of a contrast agent itself requires a certain time span, time point TP0 preferably defines the time point at which the administration is completed, i.e. at which contrast agent is completely introduced into the examination object. At time point TP1, the signal intensity of the contrast agent in the liver arteries (A) reaches its maximum. At time point TP2, the curves of the signal intensities for the liver arteries (A) and the liver veins (V) intersect. At time point TP3, the signal intensity of the contrast agent in the liver veins (V) passes through its maximum. At time point TP4, the curves of the signal intensities for the liver arteries (A) and the healthy liver cells (L) intersect. At time point TP5, the concentrations in the liver arteries (A) and the liver veins (V) have dropped to a level at which they no longer cause a measurable contrast enhancement.

During the contrast-enhanced magnetic resonance imaging examination, multiple magnetic resonance images of the liver of the patient or of part of the liver of the patient are generated. Such magnetic resonance images are referred to as representations in this description. They represent the liver or part of the liver of the patient before and/or after the administration of a contrast agent. The representations can be representations in real space or representations in frequency space.

In magnetic resonance imaging, the raw data usually arise as so-called k-space data owing to the measurement method. Said k-space data are a depiction (representation) of an examination region in frequency space. Such k-space data can be converted into a representation in real space by means of inverse Fourier transform. Conversely, representations in real space can be converted by means of Fourier transform into a representation in frequency space (also referred to as spatial frequency space or Fourier space or frequency domain or Fourier representation).

The actions described in this description are preferably carried out with representations in real space.

A representation of an examination region (e.g., of the liver) in real space is the representation that is more familiar for humans; it is more easily graspable (more understandable) for humans. For such a representation in real space, the term “image” is also usually used.

A representation in the context of the present invention can be a two-dimensional, three-dimensional or higher-dimensional representation. Usually, two-dimensional tomograms (slice images) are present or a stack of two-dimensional tomograms (slice images) are present.

The representations are usually present in digital form. The term “digital” means that the representations can be processed by a machine, generally a computer system. “Processing” is understood to mean the known methods for electronic data processing (EDP). An example of a customary format for a digital representation is the DICOM format (DICOM: Digital Imaging and Communications in Medicine)—an open standard for storing and exchanging information in medical image-data management.

In the interests of simpler illustration, the invention will be elucidated at some points in the present description on the basis of the presence of two-dimensional images, without any wish, however, to restrict the invention to two-dimensional images. It is clear to a person skilled in the art how it is possible to apply what is respectively described to stacks of two-dimensional images, to 3D images or to representations in frequency space (see, for example, M. Reisler, W. Semmler: Magnetresonanztomographie [Magnetic resonance imaging], Springer Verlag, 3rd edition, 2002, ISBN: 978-3-642-63076-7).

Digital images can be present in various formats. For example, digital images can be coded as raster graphics. Raster graphics consist of a grid arrangement of so-called picture elements (pixel) or volume elements (voxel), to which a colour or a grey value is assigned in each case. The main features of a 2D raster graphic are therefore the image size (width and height measured in pixels, also informally called image resolution) and the colour depth. A colour is usually assigned to a picture element of a digital image file. The colour coding used for a picture element is defined, inter alia, in terms of the colour space and the colour depth. The simplest case is a binary image, in which a picture element stores a black-and-white value. In the case of an image, the colour of which is defined in terms of the so-called RGB colour space (RGB stands for the primary colours red, green and blue), each picture element consists of three subpixels, a subpixel for the colour red, a subpixel for the colour green and a subpixel for the colour blue. The colour of a picture element arises through the superimposition (additive blending) of the colour values of the subpixels. The colour value of a subpixel can, for example, be divided into 256 colour nuances, which are called tonal values and usually range from 0 to 255. The colour nuance “0” of each colour channel is the darkest. If all three channels have the tonal value 0, the corresponding picture element appears black; if all three channels have the tonal value 255, the corresponding picture element appears white. When carrying out the present invention, digital images are subjected to certain operations. In this connection, the operations affect predominantly the picture elements, or the tonal values of the individual picture elements (pixel or voxel). There exists a multiplicity of possible digital image formats and colour codings. For simplification, it is assumed in this description that the present images are grey-scale raster graphics having a specific number of picture elements, with each picture element being assigned a tonal value indicating the grey value of the image. However, this assumption is not in any way to be understood as limiting. It is clear to a person skilled in the art of image processing how the teaching of said description can be applied to image files which are present in other image formats and/or in which the colour values are coded differently.

During the contrast-enhanced magnetic resonance imaging examination, a plurality of representations of the liver or part of the liver of a patient is generated.

The plurality of representations comprises at least one representation which represents the liver or the part of the liver during the portal venous phase and at least one representation which represents the liver or the part of the liver during the transitional phase.

Preference is given to further generating at least one representation of the liver or the part of the liver before the administration of the hepatobiliary contrast agent (before TP0) (native image) and/or at least one representation of the liver or the part of the liver in the arterial phase.

In one embodiment, at least the following images are generated:
at least one first representation, wherein the at least one first representation represents the liver or part of the liver during the time span from TP1 to TP3,
at least one second representation, wherein the at least one second representation represents the liver or the part of the liver during the time span from TP3 to TP5,
   wherein the time points TP0 are depicted in FIG. 1 and elucidated in the description in relation to FIG. 1.

In another embodiment, at least the following images are generated:
at least one first representation, wherein the at least one first representation represents the liver or part of the liver of a patient during the time span from TP0 to TP3,
at least a second representation, wherein the at least one second representation represents the liver or the part of the liver of the patient during the time span from TP2 to TP5,
at least a third representation, wherein the at least one third representation represents the liver or the part of the liver of the patient during the time span TP4 to 5 minutes after TP0, wherein the time points TP0 are depicted in FIG. 1 and elucidated in the description in relation to FIG. 1.

In another embodiment, at least the following images are generated:
at least one first representation, wherein the at least one first representation represents the liver or part of the liver of a patient during the time span from TP0 to TP1, and
at least one second representation, wherein the at least one second representation represents the liver or part of the liver of a patient during the time span from TP1 to TP2, and
at least one third representation, wherein the at least one third representation represents the liver or part of the liver of a patient during the time span from TP2 to TP3, and
at least one fourth representation, wherein the at least one fourth representation represents the liver or part of the liver of a patient during the time span from TP3 to TP4, and
at least one fifth representation, wherein the at least one fifth representation represents the liver or the part of the liver of the patient during the time span TP4 to TP5 seconds after TP0, and/or
at least one sixth representation, wherein the at least one sixth representation represents the liver or the part of the liver of the patient during the time span TP5 to 5 minutes after TP0,
   wherein the time points TP0 are depicted in FIG. 1 and elucidated in the description in relation to FIG. 1.

The generated representations are fed to the computer system according to the invention, which is configured to automatically analyse the representations.

The term “automatically” means without human assistance.

The analysis identifies a region or multiple regions in the liver of the patient, in which there is a washout of contrast agent in the portal venous phase and/or the transitional phase.

The washout can be identified in various ways.

In one embodiment, what are identified are those regions in the liver in which contrast agent leads in the portal venous phase and/or the transitional phase to a lower contrast enhancement than in a reference tissue. The lower contrast enhancement in the portal venous and/or transitional phase is also referred to as hypoenhancement. Hypoenhancement is the lower signal intensity in comparison with a reference tissue. In this embodiment, the reference tissue used is preferably a tissue which does not comprise hepatocytes. A suitable reference tissue is, for example, muscle tissue. An extracellular or a hepatobiliary contrast agent can be used for contrast enhancement. Preference is given to using a hepatobiliary contrast agent.

In a further embodiment, what are identified are those regions in the liver in which the contrast enhancement in the portal venous phase and/or the transitional phase drops more rapidly than in the reference tissue. In this embodiment too, the reference tissue used is preferably a tissue which does not comprise hepatocytes. A suitable reference tissue is, for example, muscle tissue. What is ascertained is the temporal gradient of signal intensity and what are identified are those regions in which the temporal gradient of signal intensity is negative (decrease in signal intensity with increase in time) and in which the absolute value of the temporal gradient is greater than the absolute value of the negative temporal gradient of signal intensity in the reference tissue. The contrast agent used can be an extracellular or a hepatobiliary contrast agent. Preference is given to using a hepatobiliary contrast agent.

In a further embodiment, what are identified are those regions in the liver in which the absolute value of the gradient of decreasing contrast enhancement in the portal venous phase and/or the transitional phase is greater than the gradient of increasing contrast enhancement in healthy liver tissue. In this embodiment, the reference tissue used is the healthy liver tissue. The contrast agent used is preferably a hepatobiliary contrast agent, which is selectively taken up by healthy liver tissue, where it leads in the portal venous and/or the transitional phase to a gradually rising signal intensity. What is thus present in the healthy liver tissue is a positive temporal gradient of signal intensity in the portal venous phase and/or the transitional phase. What are identified are those regions in which the signal intensity decreases in the portal venous phase and/or the transitional phase, wherein the absolute value of the decrease (the absolute rate of the decrease) is greater than the absolute value of the increase in signal intensity in the healthy liver tissue.

It is conceivable to combine the stated embodiments with one another. It is conceivable that identification is made of those regions in the liver in which contrast agent leads in the portal venous phase and/or the transitional phase to a lower contrast enhancement than in a first reference tissue and in which the contrast enhancement in the portal venous phase and/or the transitional phase drops more rapidly than in a second reference tissue and/or in which the absolute value of the gradient of the decreasing contrast enhancement in the portal venous phase and/or the transitional phase is greater than the gradient of the increasing contrast enhancement in healthy liver tissue. The first and/or the second reference tissue can be, for example, muscle tissue or healthy liver tissue.

In one embodiment, the present invention is used for identifying hepatocellular carcinomas. In this embodiment, one region or multiple regions is/are identified which show both a hyperenhancement in the arterial phase and a washout in the portal venous phase and/or the transitional phase.

Hyperenhancement is present when a region exhibits a higher signal intensity in comparison with a reference tissue (see, for example, M. Kim et al.: Identification of Arterial Hyperenhancement in CT and MRI in Patients with Hepatocellular Carcinoma: Value of Unenhanced Images, Korean Journal of Radiology 2019, 20(2), 236-245). The reference tissue used for detecting hyperenhancement in the arterial phase is preferably healthy liver tissue. The contrast agent used can be an extracellular or a hepatobiliary contrast agent. Preference is given to using a hepatobiliary contrast agent. Preferably, the embodiment for identification of a hepatocellular carcinoma comprises the following steps:
receiving a plurality of representations of the liver or part of the liver of a patient,
   wherein the representations are the result of a contrast-enhanced magnetic resonance imaging examination of the liver or the part of the liver,
   wherein at least one first representation represents the liver or the part of the liver during an arterial phase, wherein at least one second representation represents the liver or the part of the liver during a portal venous phase, wherein at least one third representation represents the liver or the part of the liver during a transitional phase,
analysing the at least one first, second and third representation, wherein a region or multiple regions of the liver are identified in which contrast agent leads in the arterial phase to a higher contrast enhancement than in a first reference tissue and in which contrast agent leads in the portal venous and/or transitional phase to a lower contrast enhancement than in a second reference tissue,
outputting a representation of the liver or the part of the liver, wherein in the representation the identified region is highlighted or the identified regions are highlighted.

As already described, the first reference tissue is preferably healthy liver tissue and the second reference tissue is preferably muscle tissue.

In a further embodiment, the method for identifying a hepatocellular carcinoma comprises the steps of:
receiving a plurality of representations of the liver or part of the liver of a patient,
   wherein the representations are the result of a contrast-enhanced magnetic resonance imaging examination of the liver or the part of the liver,
   wherein at least one first representation represents the liver or the part of the liver during an arterial phase, wherein at least one second representation represents the liver or the part of the liver during a portal venous phase, wherein at least one third representation represents the liver or the part of the liver during a transitional phase,
analysing the received representation, wherein a region or multiple regions of the liver is/are identified which are characterized by the following features:
   contrast agent leads in the arterial phase to a greater rise in signal than in a first reference tissue, and
   the contrast enhancement in the portal venous phase and/or the transitional phase drops more rapidly than in a second reference tissue, wherein the second reference tissue does not comprise hepatocytes, and/or
   the absolute value of the gradient of the decreasing contrast enhancement in the portal venous phase and/or the transitional phase is greater than the absolute value of the gradient of the increasing contrast enhancement in healthy liver tissue.

The first reference tissue is preferably healthy liver tissue and the second reference tissue is preferably muscle tissue.

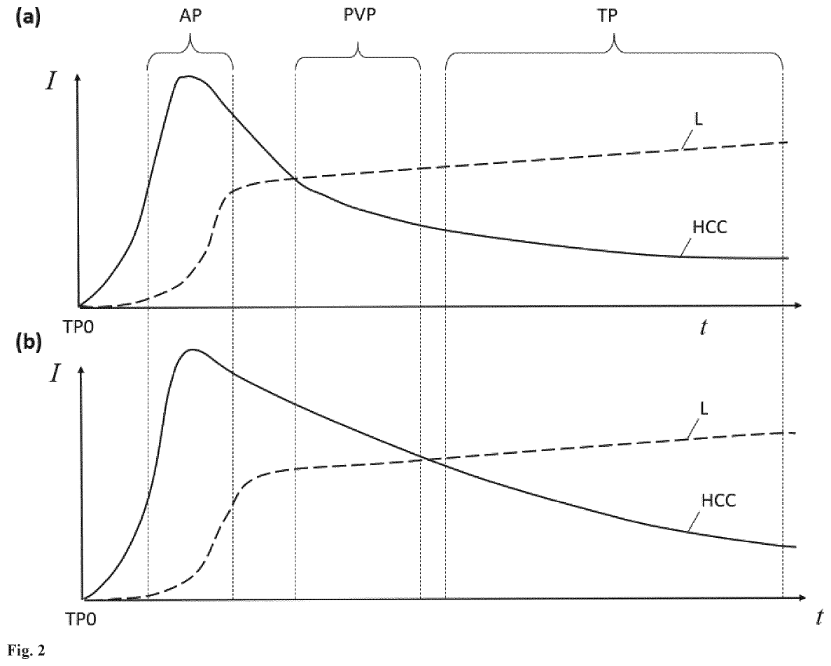

In FIG. 2 (a) and FIG. 2 (b), the terms hyperenhancement and hypoenhancement are illustrated using graphs. In FIG. 2 (a) and FIG. 2 (b), the intensity I of the signals for two different regions in the liver of a patient, which were measured during MRI examination, is depicted as a function of time t. The solid curves, which are provided with the reference sign HCC, each show the dynamic profile of the intensity I of a hepatocellular carcinoma; the dashed curves, which are provided with the reference sign L, show the dynamic profile of the intensity I of healthy liver tissue. The reference signs AP, PVP and TP indicate the arterial phase (AP), the portal venous phase (PVP) and the transitional phase (TP). Time point TP0 indicates in each case the time point of completion of the administration of a hepatobiliary contrast agent. After the administration, the intensities of the MRI signals attributable to the hepatocellular carcinomas rise sharply. The intensities of the MRI signals attributable to healthy liver tissue likewise rise—though less sharply: there is hyperenhancement in the case of HCC tissue compared to the healthy liver tissue in the arterial phase AP.

After a maximum has been passed through, the intensities of the MRI signals attributable to the tissue of the hepatocellular carcinoma drop; more rapidly in the case of the curve in FIG. 2 (a) than in the case of the curve in FIG. 2 (b). The intensities of the MRI signals attributable to healthy liver tissue rise further even after the end of the arterial phase AP; but more slowly than in the arterial phase: this indicates the uptake of contrast agent in the liver cells.

In FIG. 2 (a), the solid curve (HCC) drops in the portal venous phase PVP below the dashed curve (L): the signal enhancement in the hepatocellular carcinoma is lower than the signal enhancement in the healthy liver tissue. There is hypoenhancement in the case of the HCC tissue compared to the healthy liver tissue in the portal venous phase.

In the case of the hepatocellular carcinoma in FIG. 2 (b), the hypoenhancement does not occur until later. It is only after the end of the portal venous phase that the solid line in FIG. 2 (b) falls below the dashed line in FIG. 2 (b). Hypoenhancement does not occur until after the end of the portal venous phase.

According to the invention, the presence of a hepatocellular carcinoma is indicated when there is the presence in the lesion of a hyperenhancement compared to a first reference tissue in the arterial phase, and when there is the presence in the lesion of hypoenhancement compared to a second reference tissue in the portal venous phase or in the transitional phase or when the contrast enhancement in the portal venous phase and/or the transitional phase drops more rapidly than in a second reference tissue, wherein the second reference tissue does not comprise hepatocytes, and/or the absolute value of the gradient of the decreasing contrast enhancement in the portal venous phase and/or the transitional phase is greater than the gradient of the increasing contrast enhancement in healthy liver tissue.

To assess whether hyperenhancement and/or hypoenhancement are present, the grey values of the pixels or voxels of the real-space representations (of the 2D images or 3D images) of lesions and reference tissue(s) can be analysed.

In order to be able to make a statement about a region as to whether said region exhibits a hyperenhancement in one time span and a hypoenhancement in another time span, the region must be unambiguously identified and retrieved in the representations representing the region in the various time spans. In other words: the assessment as to whether hyperenhancement is present in a region is done on the basis of at least one first representation representing the region after the administration of a contrast agent in the arterial phase; the assessment as to whether hypoenhancement is present in a region is done on the basis of at least one second representation representing the region after the administration of a contrast agent in the portal venous phase and/or the transitional phase; the region must thus be unambiguously determinable both in the first representation and in the second representation and the region in the at least one first representation and in the at least one second representation must be the same one.

To this end, the representations generated can be subjected to an image registration. Image registration (also called “co-registration”) is a process in digital image processing and serves to bring two or more images of the same scene, or at least similar scenes, in harmony with one another in the best possible way. One of the images is defined as the reference image and the others are called object images. In order to optimally match said object images with the reference image, a compensating transformation is calculated. The images to be registered differ from one another because they were acquired from different positions, at different time points or with different sensors.

In the case of the representations of the present invention, they were acquired at different time points.

The goal of image registration is thus to find that transformation which brings a given object image in harmony with the reference image in the best possible way. The goal is that, where possible, each pixel/voxel of an image represents the same region in the body of a patient as the pixel/voxel of a different (co-registered) image having the same coordinates.

Methods for image registration are described in the prior art (see, for example: E. H. Seeley et al.: Co-registration of multi-modality imaging allows for comprehensive analysis of tumor-induced bone disease, Bone 2014, 61, 208-216; C. Bhushan et al.: Co-registration and distortion correction of diffusion and anatomical images based on inverse contrast normalization, Neuroimage 2015, 15, 115: 269-80; US20200214619; US20090135191; EP3639272A).

Co-registration can be done for each entire representation (the entire image with all anatomical features captured in the image). It is also conceivable to restrict the co-registration to the lesions, i.e. to alter the individual representations by transformation in such a way that at least the lesions in the individual representations are represented by the corresponding pixel/voxel (wherein corresponding pixels/voxels have the same coordinates).

It is further conceivable to subject each representation to a segmentation method which detects lesions in the representations and marks them as such.

Methods for detecting and segmenting lesions are described in the prior art (see, for example: C. Krishnamurthy et al.: Snake-based liver lesion segmentation, 6th IEEE Southwest Symposium on Image Analysis and Interpretation 2004 pp. 187-191, doi: 10.1109/IAI.2004.1300971; F.-A. Maayan et al.: GAN-based synthetic medical image augmentation for increased CNN performance in liver lesion classification, Neurocomputing 2018, 321, 321-331; WO2005/106773; EP3629898A; WO2012/040410).

The region or the regions in a representation that represent reference tissue can be defined/determined by a radiologist or automatically.

It is thus conceivable that a radiologist marks in the representations of the liver or part of the liver one or more regions which act as reference region(s).

For automatic determination of one or more reference regions, use can also be made of segmentation methods (see, for example, WO2020/144134), which, for example, detect muscle tissue and/or liver tissue in the representations and define a reference region which represents the muscle tissue and/or liver tissue.

According to the teaching of the present invention, hyperenhancement is present for a region (a lesion) when the grey values of the pixels/voxels representing the region within the arterial phase lie significantly above the grey values of the pixels/voxels representing reference tissue.

Hypoenhancement is present for a region (a lesion) when the grey values of the pixels/voxels representing the region within the arterial phase lie significantly above the grey values of the pixels/voxels representing the reference tissue.

The term “significantly” means that a higher or lower grey value is a measurement result which lies beyond the error limits of the measurement system.

In principle, it is sufficient for detection of hyperenhancement to compare the grey value of a pixel/voxel representing the region with the grey value of a pixel/voxel representing a reference tissue; if the grey value of the pixel/voxel of the region is greater than the grey value of the pixel/voxel of the reference region (higher signal enhancement), hyperenhancement is present; otherwise, it is not present. Analogously, it is in principle sufficient for detection of hypoenhancement to compare the grey value of a pixel/voxel representing the region with the grey value of a pixel/voxel representing a reference tissue; if the grey value of the pixel/voxel of the region is smaller than the grey value of the pixel/voxel of the reference region (lower signal enhancement), hypoenhancement is present; otherwise, it is not present.

Preference is given to evaluating multiple pixels/voxels of the region and of the reference region. Preferably, the multiple pixels/voxels define a continuous region in one representation or in multiple representations; in other words: the multiple pixels/voxels preferably represent a continuous region in the body of the patient. If multiple pixels/voxels are used, a mean of the grey value (or some other value indicating signal intensity) can be calculated (e.g. the arithmetic mean). The comparison between a region and a reference region is then done on the basis of the respective means. Instead of or in addition to an averaging over multiple locally adjacent pixels/voxels, it is also possible to perform an averaging over multiple pixels/voxels from representations which follow one another chronologically.

In a preferred embodiment, gradients of the signal intensities are also ascertained in addition to the signal intensities. For a region, such a gradient can, for example, be obtained from two representations which represent the region at a time interval from one another. The time interval can, for example, lie within the range from 1 second to 30 seconds. The shorter the time interval, the higher the accuracy with which changes (gradients) in the signal intensity can be determined. Preferably, gradients in a phase are determined by generating a number of 2 to 5 representations which represent the liver or part of the liver during the phase (arterial phase, portal venous phase, transitional phase). If the signal intensity for a region increases from one representation to the representation following chronologically, then the gradient is positive; by contrast, if the signal intensity decreases, the gradient is negative. The size of the gradient provides information as to how strong (rapid) the increase in signal intensity or the decrease in signal intensity is. By determining one or more gradients following the arterial phase, it is, for example, possible to ascertain how rapid the washout of contrast agent in the portal venous and/or the transitional phase is for a region. This information can, for example, be used for defining one or more time points for the acquisition of one or more second (or further) representations.

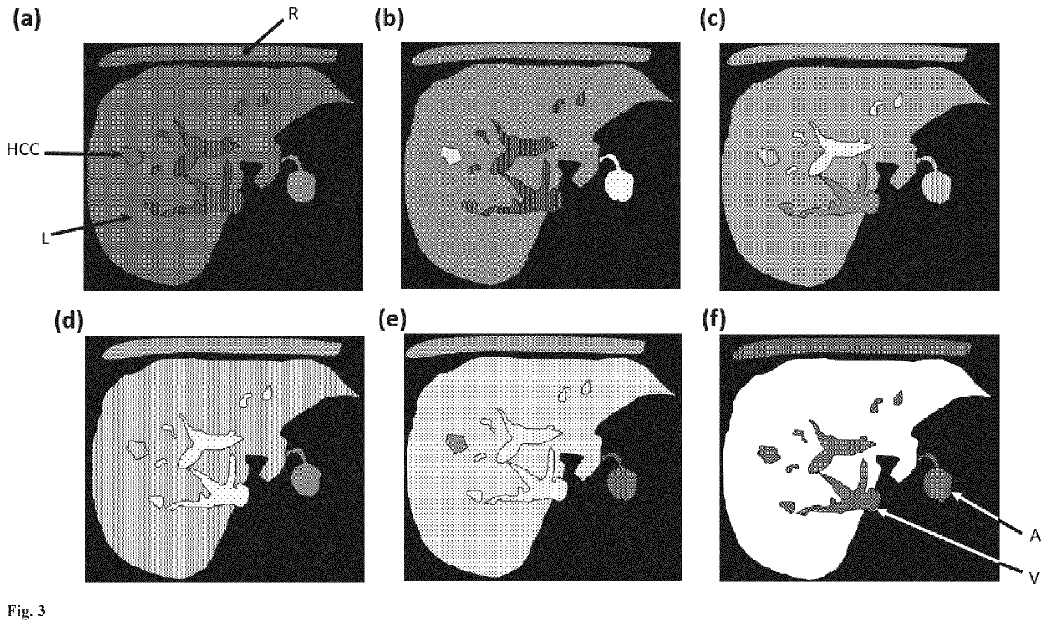

FIG. 3 shows, by way of example and in schematic form, representations of the liver of a patient. In FIGS. 3 (a), 3 (b), 3 (c), 3 (d), 3 (e) and 3 (f), the same cross section through the liver is always depicted at different time points. In addition to the liver, reference tissue (R) is also depicted; for example, it can be muscle tissue. The reference signs entered in FIGS. 3 (a) and 3 (f) apply to all of FIGS. 3 (a), 3 (b), 3 (c), 3 (d), 3 (e) and 3 (f); they are each entered only once merely for the sake of clarity.

FIG. 3 (a) shows the cross section through the liver before the intravenous administration of a hepatobiliary contrast agent (native representation). At a time point between the time points depicted by FIGS. 3 (a) and 3 (b), a hepatobiliary contrast agent was administered intravenously (e.g. into an arm vein) as a bolus. This reaches the liver via the liver artery (A) in FIG. 3 (b). Accordingly, the liver artery (A) is depicted with signal enhancement (arterial phase). A lesion, which is supplied with blood mainly via arteries, likewise stands out from the healthy liver-cell tissue (L) and the reference tissue (R) as a lighter (signal-enhanced) region. At the time point depicted in FIG. 3 (c), the contrast agent reaches the liver via the veins (V). In FIG. 3 (d), the venous blood vessels (V) stand out from the liver tissue (L) and the reference tissue (R) as light (signal-enhanced) regions (portal venous phase). At the same time, the signal intensity in the healthy liver cells (L), which are supplied with contrast agent mainly via the veins, continuously rises (FIGS. 3 (c)→3 (d)→3 (e)→3 (f)). In the hepatobiliary phase depicted in FIG. 3 (f), the liver cells (L) are depicted with signal enhancement; the blood vessels, the reference tissue and the lesion no longer have contrast agent and are accordingly depicted darkly.

The question arises as to whether the lesion (HCC) in FIG. 3 is a hepatocellular carcinoma.

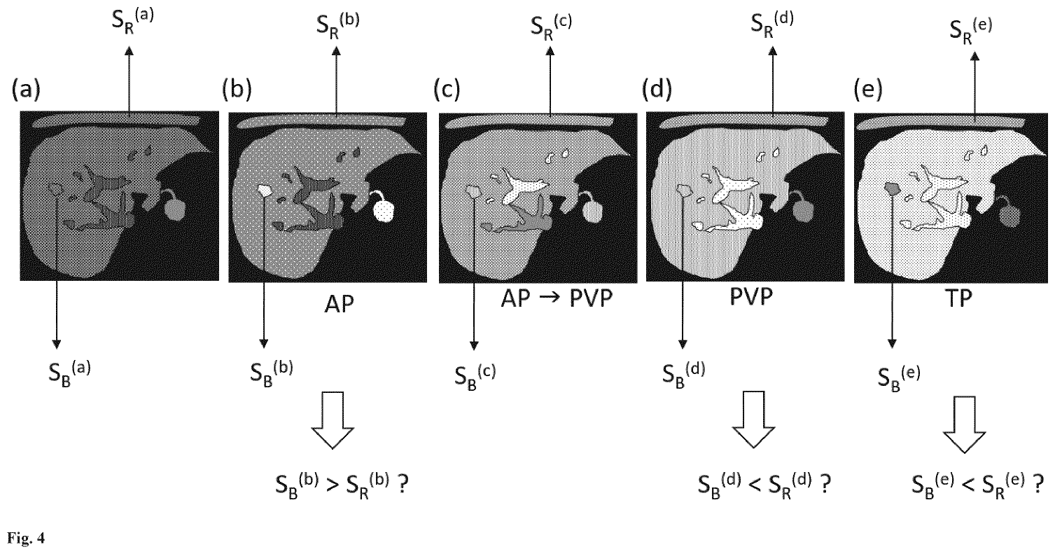

In FIG. 4, the representations shown in FIGS. 3 (a), 3 (b), 3 (c), 3 (d) and 3 (e) are depicted once more and on a smaller scale. They bear the reference signs (a), (b), (c), (d) and (e).

A signal intensity S_{B} can be ascertained for the region in the representations that represents the lesion; what can be ascertained are signal intensity S_{B}^{(a)} for representation (a), signal intensity S_{B}^{(b)} for representation (b), signal intensity S_{B}^{(c)} for representation (c), signal intensity S_{B}^{(d)} for representation (d), and signal intensity S_{B}^{(e)} for representation (e). The signal intensities can be, for example, the grey values or colour values of pixels/voxels representing the region.

Analogously, a signal intensity S_{R} can be ascertained for the region in the representations that represents the reference tissue; what can be ascertained are signal intensity S_{R}^{(a)} for representation (a), signal intensity S_{R}^{(b)} for representation (b), signal intensity S_{R}^{(c)} for representation (c), signal intensity S_{R}^{(d)} for representation (d), and signal intensity S_{R}^{(e)} for representation (e). The signal intensities can be, for example, the grey values or colour values of pixels/voxels representing the region.

In order to settle the question of whether the lesion is a hepatocellular carcinoma, signal intensities in the region representing the lesion are compared with signal intensities in the region representing the reference tissue, specifically for at least one representation during the arterial phase AP and at least one representation during the portal venous phase PVP and/or at least one representation during the transitional phase TP. Representation (b) represents the arterial phase AP. A check is made as to whether signal intensity S_{B}^{(b)} in the region representing the lesion is greater than signal intensity S_{R}^{(b)} in the region representing the reference tissue. If so, there is a first indication that the lesion is a hepatocellular carcinoma. If not, there is no need to check further signal intensities; a hepatocellular carcinoma can be ruled out.

Representation (d) represents the portal venous phase PVP. A check is made as to whether signal intensity S_{B}^{(d)} in the region representing the lesion is smaller than signal intensity S_{R}^{(d)} in the region representing the reference tissue. If so, there is a second indication that the lesion is a hepatocellular carcinoma. If the first and the second indication are present, the lesion is, according to the invention, indicated as a hepatocellular carcinoma. If only the first indication is present, but not the second indication, the transitional phase TP is looked at. Representation (e) represents the transitional phase TP. A check is made as to whether signal intensity S_{B}^{(e)} in the region representing the lesion is smaller than signal intensity S_{R}^{(e)} in the region representing the reference tissue. If S_{B}^{(e)} is smaller than S_{R}^{(e)} and the first indication is also present, what is indicated according to the invention is that the lesion is a hepatocellular carcinoma.

If it has been ascertained according to the invention for a lesion that it is a hepatocellular carcinoma, a message that indices for a hepatocellular carcinoma are present can be output. What is preferably output is a representation of the liver or part of the liver of the patient, in which the lesion in which indices for a hepatocellular carcinoma are present is marked. The relevant lesion can, for example, be marked in colour.

The invention can be carried out with the aid of a computer system.

A “computer system” is a system for electronic data processing that processes data by means of programmable computation rules. Such a system usually comprises a “computer”, the unit that comprises a processor for carrying out logical operations, and also a peripheral.

In computer technology, “peripherals” refers to all devices that are connected to the computer and are used for control of the computer and/or as input and output devices. Examples thereof are monitor (screen), printer, scanner, mouse, keyboard, drives, camera, microphone, speakers, etc. Internal ports and expansion cards are also regarded as peripherals in computer technology.

Today's computer systems are commonly subdivided into desktop PCs, portable PCs, laptops, notebooks, netbooks and tablet PCs, and so-called handhelds (e.g. smartphones); all such systems can be used for execution of the invention.

Inputs into the computer system are achieved via input means such as, for example, a keyboard, a mouse, a microphone, a touch-sensitive display and/or the like.

Outputs can be achieved on a monitor, on a printer or a data storage medium.

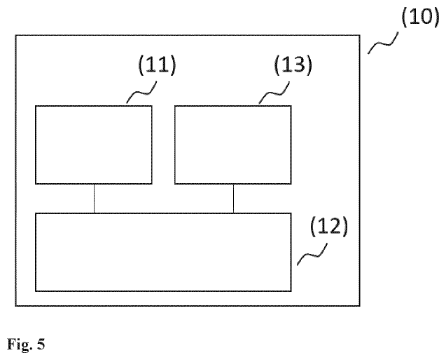

FIG. 5 shows, in schematic form and by way of example, one embodiment of the computer system according to the invention. The computer system (10) comprises a receiving unit (11), a control and calculation unit (12) and an output unit (13).

The computer system (10) according to the invention is configured to receive representations of a liver or part of the liver of a patient and to identify one or more regions in the representations that point to a hepatocellular carcinoma.

The control and calculation unit (12) serves for control of the receiving unit (11) and the output unit (13), coordination of the data and signal flows between the various units, processing of representations and ascertainment comparison of signal intensities. It is conceivable that multiple control and calculation units are present.

The receiving unit (11) serves for receiving the representations. The representations can, for example, be transmitted from a magnetic resonance imaging system or be read from a data storage medium. The magnetic resonance imaging system can be a component of the computer system according to the invention. However, it is also conceivable that the computer system according to the invention is a component of a magnetic resonance imaging system. Representations can be transmitted via a network connection or a direct connection. Representations can be transmitted via radio communication (WLAN, Bluetooth, mobile communications and/or the like) and/or via a cable. It is conceivable that multiple receiving units are present. The data storage medium, too, can be a component of the computer system according to the invention or be connected thereto, for example via a network. It is conceivable that multiple data storage media are present.

The representations possibly further data (such as, for example, information about the examination object, image-acquisition parameters and/or the like) are received by the receiving unit and transmitted to the control and calculation unit.

The control and calculation unit is configured to identify, on the basis of the received data, regions which point to a hepatocellular carcinoma.

Via the output unit (13), the results of the analysis can be displayed (e.g. on a monitor), be output (e.g. via a printer) or be stored in a data storage medium. It is conceivable that multiple output units are present.

Further embodiments of the present invention are:
1. A computer-implemented method comprising the steps of:
   receiving a plurality of representations of the liver or part of the liver of a patient who has been subjected to a contrast-enhanced magnetic resonance imaging examination, wherein at least one representation represents a liver or part of the liver of the patient during a portal venous phase and at least one representation represents the liver or the part of the liver during a transitional phase,
   identifying one or more regions in the liver in which contrast agent leads in the portal venous phase and/or the transitional phase to a lower contrast enhancement than in a reference tissue and/or the contrast enhancement in the portal venous phase and/or the transitional phase drops more rapidly than in the reference tissue, wherein the reference tissue does not comprise hepatocytes, and/or the absolute value of the gradient of the decreasing contrast enhancement in the portal venous phase and/or the transitional phase is greater than the absolute value of the gradient of the increasing contrast enhancement in healthy liver tissue,
   outputting a representation of the liver or the part of the liver, in which the identified region is highlighted or the identified regions are highlighted.
2. The method according to embodiment 1, wherein the contrast agent is a hepatobiliary contrast agent, preferably the disodium salt of gadoxetic acid.
3. The method according to either of embodiments 1 and 2, wherein the reference tissue is muscle tissue.
4. The method according to any of embodiments 1 to 3, comprising the steps of:
   receiving the plurality of representations of the liver or part of the liver of a patient, wherein at least two representations represent the liver or the part of the liver during the portal venous phase and at least two representations represent the liver or the part of the liver during the transitional phase,
   analysing the plurality of received representations and, in doing so, identifying the one region or the multiple regions in the liver in which the contrast enhancement in the portal venous phase and/or the transitional phase drops more rapidly than in the reference tissue, wherein the reference tissue does not comprise hepatocytes, and/or in which the absolute value of the gradient of the decreasing contrast enhancement in the portal venous phase and/or the transitional phase is greater than the absolute value of the gradient of the increasing contrast enhancement in healthy liver tissue,
   outputting the representation of the liver or the part of the liver, in which the identified region is highlighted or the identified regions are highlighted.
5. The method according to any of embodiments 1 to 4, further comprising the steps of:
   receiving at least one representation of the liver or the part of the liver that represents the liver or the part of the liver during an arterial phase,
   identifying one or more regions in the liver, wherein the region/regions is/are characterized by the following features:
      contrast agent leads in the arterial phase to a higher contrast enhancement than in a first reference tissue, wherein the first reference tissue is healthy liver tissue or muscle tissue, and
      contrast agent leads in the portal venous and/or transitional phase to a lower contrast enhancement than in a second reference tissue, wherein the second reference tissue is muscle tissue, and/or
      the contrast enhancement in the portal venous phase and/or the transitional phase drops more rapidly than in the second reference tissue, and/or
      the absolute value of the gradient of the decreasing contrast enhancement in the portal venous phase and/or the transitional phase is greater than the absolute value of the gradient of the increasing contrast enhancement in healthy liver tissue,
   outputting a representation of the liver or the part of the liver, in which the identified region is highlighted or the identified regions are highlighted.
6. A computer system comprising:
   a receiving unit,
   a control and calculation unit and
   an output unit,
      wherein the control and calculation unit is configured to prompt the receiving unit to receive a plurality of representations of a liver or part of the liver of a patient, wherein at least one representation represents the liver or the part of the liver during a portal venous phase and at least one representation represents the liver or the part of the liver during a transitional phase,
      wherein the control and calculation unit is configured to identify one or more regions in the liver in which contrast agent leads in the portal venous phase and/or the transitional phase to a lower contrast enhancement than in a reference tissue and/or the contrast enhancement in the portal venous phase and/or the transitional phase drops more rapidly than in the reference tissue, wherein the reference tissue does not comprise hepatocytes, and/or the absolute value of the gradient of the decreasing contrast enhancement in the portal venous phase and/or the transitional phase is greater than the absolute value of the gradient of the increasing contrast enhancement in healthy liver tissue,
      wherein the control and calculation unit is configured to prompt the output unit to output a representation of the liver or the part of the liver, in which the identified region is highlighted or the identified regions are highlighted.
7. A computer program product comprising a computer program which can be loaded into a memory of a computer, where it prompts the computer to execute the following steps:
   receiving a plurality of representations of the liver or part of the liver of the patient, wherein at least one representation represents the liver or the part of the liver during a portal venous phase and at least one representation represents the liver or the part of the liver during a transitional phase,
   identifying one or more regions in the liver in which contrast agent leads in the portal venous phase and/or the transitional phase to a lower contrast enhancement than in a reference tissue and/or the contrast enhancement in the portal venous phase and/or the transitional phase drops more rapidly than in the reference tissue, wherein the reference tissue does not comprise hepatocytes, and/or the absolute value of the gradient of the decreasing contrast enhancement in the portal venous phase and/or the transitional phase is greater than the absolute value of the gradient of the increasing contrast enhancement in healthy liver tissue,
   outputting a representation of the liver or the part of the liver, in which the identified region is highlighted or the identified regions are highlighted.
8. The use of a contrast agent in a radiological examination method, wherein the radiological examination method comprises the following steps:
   administering a contrast agent,
   generating a plurality of representations of a liver or part of the liver of a patient, wherein at least one representation represents the liver or the part of the liver during a portal venous phase and at least one representation represents the liver or the part of the liver during a transitional phase,
   identifying one or more regions in the liver in which contrast agent leads in the portal venous phase and/or the transitional phase to a lower contrast enhancement than in a reference tissue and/or the contrast enhancement in the portal venous phase and/or the transitional phase drops more rapidly than in the reference tissue, wherein the reference tissue does not comprise hepatocytes, and/or the absolute value of the gradient of the decreasing contrast enhancement in the portal venous phase and/or the transitional phase is greater than the absolute value of the gradient of the increasing contrast enhancement in healthy liver tissue,
   outputting a representation of the liver or the part of the liver, in which the identified region is highlighted or the identified regions are highlighted.
9. A contrast agent for use in a radiological examination method, wherein the radiological examination method comprises the following steps:
   administering a contrast agent,
   generating a plurality of representations of a liver or part of the liver of a patient, wherein at least one representation represents the liver or the part of the liver during a portal venous phase and at least one representation represents the liver or the part of the liver during a transitional phase,
   identifying one or more regions in the liver in which contrast agent leads in the portal venous phase and/or the transitional phase to a lower contrast enhancement than in a reference tissue and/or the contrast enhancement in the portal venous phase and/or the transitional phase drops more rapidly than in the reference tissue, wherein the reference tissue does not comprise hepatocytes, and/or the absolute value of the gradient of the decreasing contrast enhancement in the portal venous phase and/or the transitional phase is greater than the absolute value of the gradient of the increasing contrast enhancement in healthy liver tissue,
   outputting a representation of the liver or the part of the liver, in which the identified region is highlighted or the identified regions are highlighted.
10. A kit comprising a contrast agent and a computer program product according to embodiment 7.
11. The kit according to embodiment 10, wherein the contrast agent is the disodium salt of gadoxetic acid.

1: A computer-implemented method comprising:
receiving a plurality of representations, wherein the plurality of representations represents;
a liver of a patient or part of the liver of the patient, and
reference tissue of the patient,
wherein at least one representation of the plurality of representations represents the liver or the part of the liver and the reference tissue during a portal venous phase of a dynamic contrast-enhanced magnetic resonance imaging examination, and
wherein at least one representation of the plurality of representations represents the liver or the part of the liver and the reference tissue during a transitional phase of the dynamic contrast-enhanced magnetic resonance imaging examination;
identifying one or more regions in the liver;
in which contrast agent leads in the portal venous phase and/or the transitional phase to a lower contrast enhancement than in the reference tissue, and/or
in which the contrast enhancement in the portal venous phase and/or the transitional phase drops more rapidly than in the reference tissue, wherein the reference tissue does not comprise hepatocytes, and/or
in which the absolute value of the gradient of the decreasing contrast enhancement in the portal venous phase and/or the transitional phase is greater than the absolute value of the gradient of the increasing contrast enhancement in healthy liver tissue; and
outputting a representation of the liver or the part of the liver, wherein in the representation the identified region is highlighted or the identified regions are highlighted.

2: The method of claim 1, wherein the contrast agent is a hepatobiliary contrast agent.

3: The method of claim 1, wherein the reference tissue is muscle tissue.

4: The method of claim 1, comprising:
receiving the plurality of representations, wherein;
at least two representations of the plurality of representations represent the liver or the part of the liver and the reference tissue during the portal venous phase, and
at least two representations of the plurality of representations represent the liver or the part of the liver and the reference tissue during the transitional phase;
analysing analyzing the plurality of received representations to identify the one region or the multiple regions in the liver:
in which the contrast enhancement in the portal venous phase and/or the transitional phase drops more rapidly than in the reference tissue, wherein the reference tissue does not comprise hepatocytes, and/or
in which the absolute value of the gradient of the decreasing contrast enhancement in the portal venous phase and/or the transitional phase is greater than the absolute value of the gradient of the increasing contrast enhancement in healthy liver tissue; and
outputting the representation of the liver or the part of the liver, wherein in the representation the identified region is highlighted or the identified regions are highlighted.

5: The method of claim 1, further comprising:
receiving at least one representation, wherein the at least one representation represents the liver or the part of the liver and a first and/or second reference tissue during an arterial phase of the dynamic contrast-enhanced magnetic resonance imaging examination;
identifying one or more regions in the liver, wherein the region/regions is/are characterized by the following features:
contrast agent leads in the arterial phase to a higher contrast enhancement than in the first reference tissue, wherein the first reference tissue is healthy liver tissue or muscle tissue, and
contrast agent leads in the portal venous and/or transitional phase to a lower contrast enhancement than in the second reference tissue, wherein the second reference tissue is muscle tissue, and/or
the contrast enhancement in the portal venous phase and/or the transitional phase drops more rapidly than in the second reference tissue, and/or
the absolute value of the gradient of the decreasing contrast enhancement in the portal venous phase and/or the transitional phase is greater than the absolute value of the gradient of the increasing contrast enhancement in healthy liver tissue; and
outputting a representation of the liver or the part of the liver, wherein in the representation the identified region is highlighted or the identified regions are highlighted.

6: A computer system comprising:
a receiving unit,
a control and calculation unit, and
an output unit,
wherein the control and calculation unit is configured to prompt the receiving unit to receive a plurality of representations, wherein the plurality of representations represents
a liver of a patient or part of the liver of the patient, and
reference tissue of the patient,
wherein at least one representation of the plurality of representations represents the liver or the part of the liver and the reference tissue during a portal venous phase of a dynamic contrast-enhanced magnetic resonance imaging examination, and
wherein at least one representation of the plurality of representations represents the liver or the part of the liver and the reference tissue during a transitional phase of the dynamic contrast-enhanced magnetic resonance imaging examination;
wherein the control and calculation unit is configured to identify one or more regions in the liver:
in which contrast agent leads in the portal venous phase and/or the transitional phase to a lower contrast enhancement than in the reference tissue, and/or
in which the contrast enhancement in the portal venous phase and/or the transitional phase drops more rapidly than in the reference tissue, wherein the reference tissue does not comprise hepatocytes, and/or
in which the absolute value of the gradient of the decreasing contrast enhancement in the portal venous phase and/or the transitional phase is greater than the absolute value of the gradient of the increasing contrast enhancement in healthy liver tissue; and
wherein the control and calculation unit is configured to prompt the output unit to output a representation of the liver or the part of the liver, wherein in the representation the identified region is highlighted or the identified regions are highlighted.

7: A non-transitory computer readable storage medium storing instructions that, when executed by one or more processors of a computer system, cause the computer system to:
receive a plurality of representations, wherein the plurality of representations represents:
a liver of a patient or part of the liver of the patient, and
reference tissue of the patient,
wherein at least one representation of the plurality of representations represents the liver or the part of the liver and the reference tissue during a portal venous phase of a dynamic contrast-enhanced magnetic resonance imaging examination, and
wherein at least one representation of the plurality of representations represents the liver or the part of the liver and the reference tissue during a transitional phase of the dynamic contrast-enhanced magnetic resonance imaging examination;
identify one or more regions in the liver:
in which contrast agent leads in the portal venous phase and/or the transitional phase to a lower contrast enhancement than in the reference tissue, and/or
in which the contrast enhancement in the portal venous phase and/or the transitional phase drops more rapidly than in the reference tissue, wherein the reference tissue does not comprise hepatocytes, and/or
in which the absolute value of the gradient of the decreasing contrast enhancement in the portal venous phase and/or the transitional phase is greater than the absolute value of the gradient of the increasing contrast enhancement in healthy liver tissue; and
outputting output a representation of the liver or the part of the liver, wherein in the representation the identified region is highlighted or the identified regions are highlighted.

8: The use of a contrast agent in a dynamic contrast-enhanced magnetic resonance imaging examination method, wherein the examination method comprises:
administering the contrast agent;
generating a plurality of representations wherein the plurality of representations represents;
a liver of a patient or part of the liver of the patient, and
reference tissue of the patient,
wherein at least one representation of the plurality of representations represents the liver or the part of the liver and the reference tissue during a portal venous phase of a dynamic contrast-enhanced magnetic resonance imaging examination, and
wherein at least one representation of the plurality of representations represents the liver or the part of the liver and the reference tissue during a transitional phase of the dynamic contrast-enhanced magnetic resonance imaging examination;
identifying one or more regions in the liver;
in which contrast agent leads in the portal venous phase and/or the transitional phase to a lower contrast enhancement than in the reference tissue, and/or
in which the contrast enhancement in the portal venous phase and/or the transitional phase drops more rapidly than in the reference tissue, wherein the reference tissue does not comprise hepatocytes, and/or
in which the absolute value of the gradient of the decreasing contrast enhancement in the portal venous phase and/or the transitional phase is greater than the absolute value of the gradient of the increasing contrast enhancement in healthy liver tissue; and
outputting a representation of the liver or the part of the liver, wherein in the representation the identified region is highlighted or the identified regions are highlighted.

9: A contrast agent for use in a dynamic contrast-enhanced magnetic resonance imaging examination method, wherein the examination method comprises:
administering the contrast agent;
generating a plurality of representations wherein the plurality of representations represents;
a liver of a patient or part of the liver of the patient, and
reference tissue of the patient,
wherein at least one representation of the plurality of representations represents the liver or the part of the liver and the reference tissue during a portal venous phase of a dynamic contrast-enhanced magnetic resonance imaging examination, and
wherein at least one representation of the plurality of representations represents the liver or the part of the liver and the reference tissue during a transitional phase of the dynamic contrast-enhanced magnetic resonance imaging examination;
identifying one or more regions in the liver:
in which contrast agent leads in the portal venous phase and/or the transitional phase to a lower contrast enhancement than in the reference tissue, and/or
in which the contrast enhancement in the portal venous phase and/or the transitional phase drops more rapidly than in the reference tissue, wherein the reference tissue does not comprise hepatocytes, and/or
in which the absolute value of the gradient of the decreasing contrast enhancement in the portal venous phase and/or the transitional phase is greater than the absolute value of the gradient of the increasing contrast enhancement in healthy liver tissue; and
outputting a representation of the liver or the part of the liver, wherein in the representation the identified region is highlighted or the identified regions are highlighted.

10: A kit comprising a contrast agent and the non-transitory computer readable storage medium of 7.

11: The kit of claim 10, wherein the contrast agent is a hepatobiliary contrast agent.

12: The kit of claim 10, wherein the contrast agent is a disodium salt of gadoxetic acid.

13: The method of claim 2, wherein the contrast agent is a disodium salt of gadoxetic acid.
